(19) United States
(12) Patent Application Publication
(10) Pub. No.: US 2017/0112384 A1
(43) Pub. Date: Apr. 27, 2017

(54) Optical Laser Catheter for Intracorporeal Diagnostic and Treatment Based Photoacoustic Spectroscopy

(71) Applicants: Saher Maswadi, San Antonio, TX (US); Randolph Glickman, San Antonio, TX (US); Kelly Nash, San Antonio, TX (US); John Taboada, San Antonio, TX (US)

(72) Inventors: Saher Maswadi, San Antonio, TX (US); Randolph Glickman, San Antonio, TX (US); Kelly Nash, San Antonio, TX (US); John Taboada, San Antonio, TX (US)

(73) Assignee: The Board of Regents of the University of Texas System, Austin, TX (US)

(21) Appl. No.: 15/298,752

(22) Filed: Oct. 20, 2016

Related U.S. Application Data
(60) Provisional application No. 62/244,372, filed on Oct. 21, 2015.

(51) Int. Cl. A61B 5/00; A61B 18/24; A61B 8/12; A61B 1/06; A61B 1/07

(52) U.S. Cl. CPC A61B 5/0095; A61B 1/063; A61B 1/07; A61B 5/6852; A61B 8/12; A61B 18/24; A61B 2017/00061

(57) ABSTRACT

Certain embodiments are directed to an interventional device and methods of use of an interventional device comprising all-optical photoacoustic imaging and optionally further comprising at least one medical treatment device.

This application claims priority to U.S. Provisional Patent Application Ser. No. 62/244,372 filed Oct. 21, 2015, which is incorporated herein by reference in its entirety.

### BACKGROUND

Artery disease affects more than 16 million Americans, making it the most common form of vascular and heart disease. Artery disease often results from a condition known as atherosclerosis, which results from plaque forming inside arteries supplying blood to the heart. Plaque is composed of cholesterol, fatty compounds, calcium, and a blood-clotting material called fibrin. As plaque builds the artery narrows making it more difficult for blood to flow to the heart.

Various imaging modalities can be applied to image vessel diseases. Angiography is the most widely used modality to detect the systematic distribution and the degree of stenosis of vessels. However, in angiography, the arbitrary projection of vessels onto a 2D plane may misrepresent the true vessel lumen narrowing, therefore leading to the misjudgment of the plaque distribution (Nissen, Am J Cardiol., 87(4A) 15A-20A, 2001; Nissen and Yock, Circulation 103(4) 604-16, 2001). Catheter-based intravascular ultrasound (IVUS) is one of the emerging imaging tools of the clinical evaluation of atherosclerosis (Nicholls et al., Am Heart J, 152(1) 67-74, 2006; Lee et al., Am J Cardiol, 105(10) 1378-84, 2010). An IVUS catheter, having a diameter of 1-1.4 mm (French gauge 3 to 4), is inserted into a vessel lumen to image the vessel wall using a high frequency ultrasound transducer at the tip of the catheter. Pressure waves are generated in a specific direction and the backscattered ultrasound waves are received by the transducer. To form cross-sectional images of the vessel, a single element transducer needs to be mechanically rotated. IVUS can image the lumen geometry and the structure of the wall with a resolution equal to 100 μm. However, histopathological information obtained with intravascular ultrasound (IVUS) imaging is limited by the lack of specificity for different compounds of soft tissues (Palmer et al., Eur Heart J 20(23) 1701-06, 1999). In addition IVUS catheters are not coupled to a treatment catheter (e.g., laser based catheter).

Photoacoustic (PA) imaging is an emerging technology that couples optics and acoustics into one modality. In photoacoustic imaging, a laser pulse with nano- to picosecond duration is emitted onto the tissue (an excitation pulse). After absorbing the laser energy, the tissue generates broadband photoacoustic (or optoacoustic) signals due to its fast thermal expansion (Zhang et al., Biomed Opt Express 3(7) 1662-29, 2012; Su et al., Opt Express 17(22) 19894-901, 2009; Page et al., Proc. SPIE 789931, 2011; Ermilov et al., J Biomed Opt 14(2) 24007, 2009; Page et al., Applied Spectroscopy Journal MS (11-06562) 2012). The major advantage of PA imaging is the ability to selectively tune signal amplitude, this allows for the spectroscopic capability to target absorption coefficients of tissue (Page et al., Proc. SPIE 789931, 2011; Page et al., Proc. SPIE 76290E, 2010). Under known laser fluence and constant tissue temperature, photoacoustic imaging can map the optical absorption property of the tissue. One of the successful applications of PA imaging is tomography of blood vessels because of their high optical absorption contrast in the visible wavelength region (Kim et al., Radiology 255(2) 442-50, 2010; Hoelen et al., Opt Lett, 23(8) 648-50, 1998). PA spectroscopy can provide functional information such as blood oxygenation or the velocity of the blood flow by using multi wavelength photoacoustic imaging or frequency analysis of the PA signals (Yao et al., Opt Lett 35(9) 1419-21, 2010; Petrov et al., Anesthesiology 102(1) 69-75, 2005).

There remains a need for additional therapeutic devices that can access, image, and/or treat using a single device.

### SUMMARY

There is a clinical need to characterize the composition of plaques and identify those plaques vulnerable to laser ablation, as well monitoring the progress or results of treatment. In certain aspects, systems and devices described herein can be used for imaging and/or imaging and treating the vascular system and other tissues. Clinical decisions regarding the appropriate use of therapeutic and interventional strategies depend on the type of plaques or tissues to be treated.

Certain embodiments are directed to an intravascular optical photoacoustic (PA) imaging device. In certain aspects, the imaging device employs one or more optical fibers. In certain embodiments, a PA probe is coupled to an optical fiber, a laser source, and/or a detector. In certain aspects a laser source is a tunable laser source. In certain aspects the detector is an interferometer.

In certain embodiments, the intravascular device has a proximal end coupled to a laser source and a detector. The device can also comprise a distal end configured to deliver optical beams and to receive acoustic waves. In certain aspects, the acoustic waves are detected by probe beam deflection. In further aspects, an intravascular device is configured to transmit an optical probe beam via a first optical fiber and an excitation beam and/or therapeutic beam using a second fiber. In further aspects, the first optical fiber is positioned along the longitudinal axis or core of the device. In still further aspects, the optical fiber can be off center relative to the axis of the device and run parallel to the longitudinal axis of the device. In certain aspects the fiber core can be a single mode or multi-mode optical fiber.

The distal imaging probe may comprises a lens, coupling medium, and/or a reflector. The lens may be configured to direct light to a reflector capping the distal end of a probe chamber. The probe chamber may be made of or filled with an acoustic coupling medium. In some aspects, the coupling medium may transmit acoustic waves that strike the probe, which in turn propagates through the coupling medium and alters the refractive index of the coupling medium. The probe chamber may be configured to be transparent to an excitation beam to illuminate a target and generate acoustic waves. The probe chamber may be made of, but not limited to, glass, mylar, acrylic, or other suitable polymers. The probe chamber can be solid or configured to contain another medium such as, but not limited to, water or a low attenuating fluid of optical quality (i.e., a fluid filled probe chamber). The reflector is configured to reflect the probe beam back to the distal end of the device once it has passed through the coupling medium.

In some embodiments, a probe as described herein may be used as a diagnostic or imaging tool. In certain aspects, the probe is configured for intracorporeal applications. Applications may include identifying tissue type based on absorption spectrum using photoacoustic/optoacoustic (PA/OA) spectroscopy. In certain aspects, the probe is configured as an all-optical sensor to detect acoustic signals, which can accommodate rotation of the probe beam, excitation beam, or both without physically rotating the device. In certain aspects, the fiber system is configured to image 360 degrees with no need for physical rotation of the catheter. In certain embodiments, a PA imaging probe is incorporated into an optoacoustic (OA) catheter. Such OA catheters may be configured for use during surgical treatments such as laser treatment of plaque within vessels. In some aspects, a combination system (diagnostic/therapeutic) may be converted from diagnostic to treatment and vice versa during an intravascular surgery. Thus, such a device may characterize a target tissue or plaques, treat the target tissue or plaque, and evaluate post-treatment results. In certain embodiments, the probe is configured to be a guide sensor for a treatment catheter.

In some aspects, the probe beam may be used for tissue imaging and tissue type identification. As one non-limiting example, the visible and near IR absorption coefficient of lipid or fatty acid differs from hemoglobin; thus, plaques formed within a vessel can be characterized from PA imaging. In some aspects, a treatment beam may be a laser beam that may be used to cut or destroy a target. In some aspects, the treatment beam is an ablation beam. In some aspects, the treatment beam is capable of cutting or destroying plaques formed within a vessel and/or tissue. In certain aspects, the heat generated by the laser is used to destroy the target. In some aspects, the electromagnetic radiation from the laser is selectively absorbed by the target. Thus, precise and controlled destruction of the target, such as plaque or an unwanted tissue, may be obtained through the laser treatment beam.

In some aspects, laser photoacoustic spectroscopy may be used to image and provide characteristic information. Laser photoacoustic spectroscopy is a form of optical absorption spectroscopy. In some instances, a sample is irradiated by a picosecond to nanosecond laser pulse, the sample absorbs the optical energy and can convert a portion of it to acoustic energy due to its thermal expansion and emits a characteristic acoustic signal. In some aspects, an ultrasonic receiver or transducer may receive this acoustic signal and thereby obtain characteristic image and sample information.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 illustrates a treatment system comprising an interventional device.

FIG. 2 illustrates on embodiment of an interventional device coupled to an optics/optical fiber interface that is in turn coupled to a control device, an excitation/ablation laser source, and a probe beam source/detector.

FIG. 3 illustrates one configuration of the system comprising a control unit coupled to a tunable photoacoustic laser source and a treatment excimer laser as well as an optical coherence detection device.

FIG. 4 illustrates one embodiment of the device being used in to image a vessel.

FIG. 5 illustrates one configuration of a photoacoustic ultrasound probe coupled to a delivery fiber bundle.

### DESCRIPTION

Endoscopy is used to access target tissues by introduction of a probe percutaneously or through a natural orifice. Some of the clinical applications of endoscopy are the assessment of artery disease, prostate cancer, and gastrointestinal pathologies. Recent efforts combined photoacoustic intravascular devices with intravascular ultrasound (IVUS). This combination images vessels perpendicular to the axis of the probe and the probe must be rotated to construct an image. This combination can exploit the differing absorption coefficient spectra of endogenous tissue chromophores. For example, the absorption coefficient of lipid or fatty acid is significantly lower than that of hemoglobin Hb over the visible and near infrared spectra range up to 1100 nm. At around 1100 nm absorption is dominated by water rather than Hb and further around 1210 nm, a strong lipid peak becomes predominate. Concentrated lipid deposits can be exploited to image plaques by tuning light to the lipid absorption peak.

Herein, certain embodiments are directed to a spectroscopic diagnostic tool to be used in conjunction with laser treatment of plaques or lesions within vessels. Commercial vascular treatment catheters, such as the laser ablation catheter from Spectranetics (Colorado Springs, Co.), are not equipped with diagnostic or imaging tools during vessel treatment or plaque ablation. The spectroscopic diagnostic tools that can be used in conjunction with laser treatment may include a photoacoustic imaging modality based on optical absorption where contrast can be selectively enhanced for specific tissue components by tuning the excitation wavelength to the absorption of their chromophores, such as wavelength 1210 nm for lipid absorption.

Certain embodiments are directed to a small, fiber optic-based optical photoacoustic imaging probe. In certain aspects, the probe is an all-optical probe and can have a diameter of less than 0.5, 0.6, 0.7, 0.8, 0.9, or 1 mm. In certain aspects, the probe is combined in a single device with a diagnostic or treatment catheter to diagnose vessels, tissues, or portions of organs before, during, and/or after treatment. In some aspects, the photoacoustic diagnostic system is configured to generate and to sense pressure waves using optical techniques with a probe coupled to a catheter.

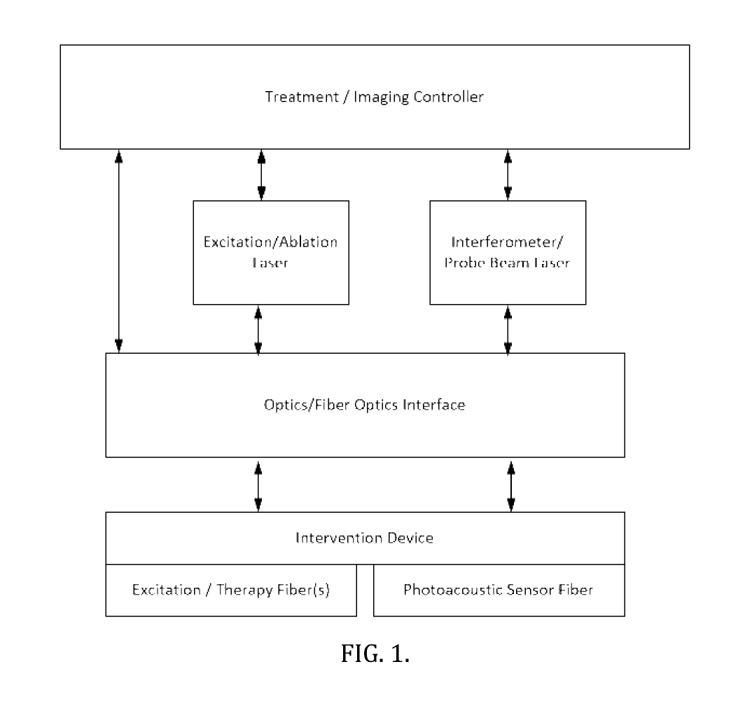

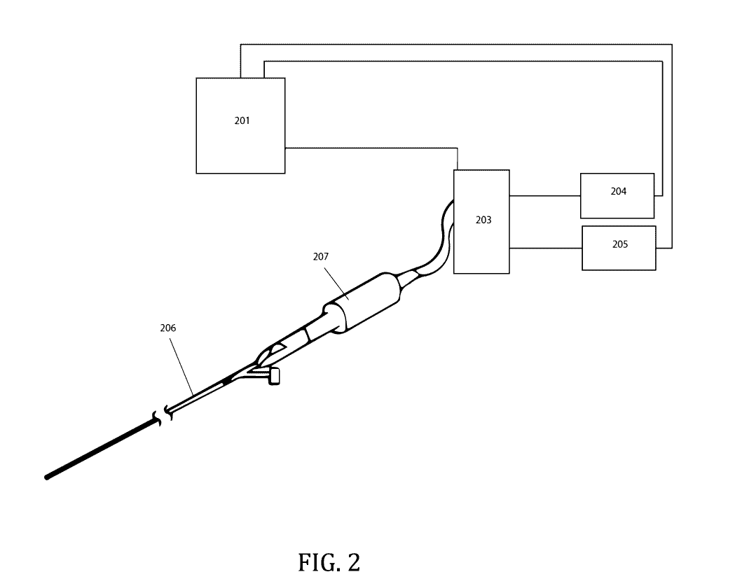

FIG. 1 and FIG. 2 diagram a non-limiting example of a basic system and set up for implementing an imaging device described herein. An interventional device (207) that may include a catheter (206), optic interface (203), and source lasers (204 and 205) may be coupled to a treatment/imaging control device (201). Also, diagrammed in FIG. 1 is a non-limiting example of an interventional device configure to have an imaging modality via a photoacoustic sensor coupled to a first optical fiber and a excitation or ablation modality via one or more second optical fibers. An optical fiber is a flexible, transparent fiber that may be made of high quality extruded glass (silica) or plastic, and may be slightly thicker than a human hair. It may function as a waveguide to transmit light between the two ends of the fiber. Optical fibers typically include a transparent core surrounded by a transparent cladding material with a lower index of refraction. Light may be kept in the core by total internal reflection. This causes the fiber to act as a waveguide.

Fibers that support many propagation paths or transverse modes are called multi-mode fibers (MMF), while those that only support a single mode are called single-mode fibers (SMF). Multi-mode fibers generally have a wider core diameter, and are used for applications where high power must be transmitted. In other aspects, two light paths can be provided by a core light path through the core of the device and circumferential light path that circumscribes or is position outside of the core light path.

Optical Fiber Optic Imaging.

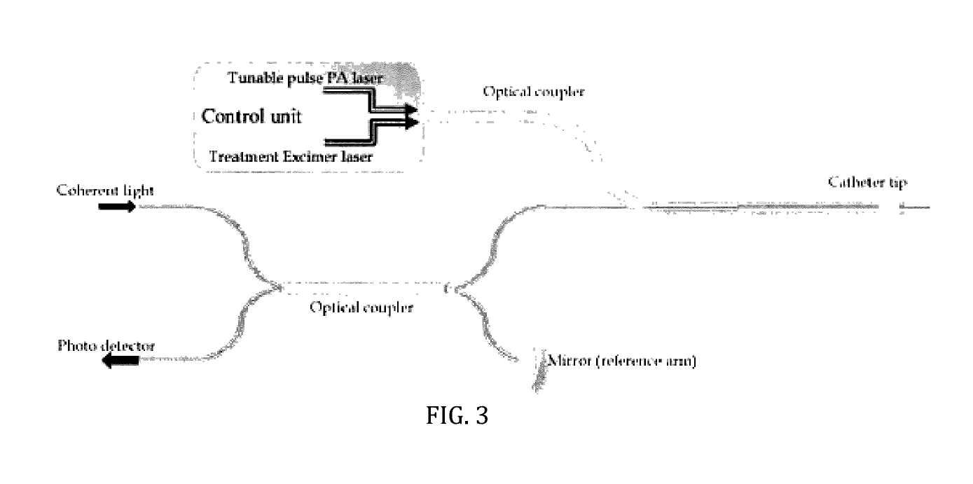

Rarely has a PA imaging or diagnostic system been evaluated in vivo. One of the challenges is to technically integrate a system of delivery and probing with commercial or FDA approved diagnostic/treatment systems while maintaining the necessary size for intravascular use. FIG. 3 illustrates a non-limiting example of a PA system based on optical fiber optics configured to deliver and to receive an acoustic signal with optical output. In certain aspects, the probe is an all-optical probe coupled to a laser source and a detector. In certain aspects, systems are designed for implementation with existing commercial fiber optic laser treatment of plaque. Optical interferometry may include a Michelson interferometer or an optical coherence interferometer configured as an ultrasound sensor.

Imaging Forward Fiber Probe Coupled with Treatment Fibers.

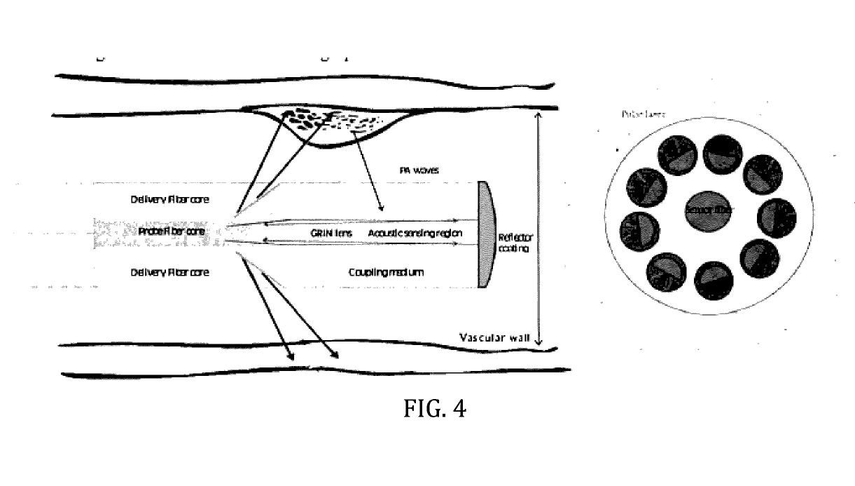

In some aspects, laser ablation catheters may be constructed of multiple optical fibers arranged around a guidewire lumen or, in the particular device shown, a sensor fiber. FIG. 4 shows a non-limiting example where the core is a sensor fiber instead of a guidewire lumen. In some embodiments, multi-fiber catheters may transmit electromagnetic energy, such as ultraviolet energy (e.g. Excimer laser), to an obstruction in the artery. The electromagnetic energy can be delivered to the tip of the laser catheter to ablate plaque, fibrous, and/or calcific regions. Currently, the guidewire is the only sensing tool provided with laser catheters to locate plaque or lesion, which are identified by mechanical feedback. However, a standard guidewire offers little actual information to the surgeon. Guidewires also tend to fail to identify complications because no imaging feedback is provided. Non-limiting examples of when a lack of imaging feedback may cause a failure to identify a complication includes (i) contacting rounded or eccentric occlusion stumps that deflect the guidewire to a subintimal passage, (ii) repeated deflection into a large collateral branch flush with the occlusion stump, or (iii) contacting calcification that obstructs completion of the guidewire passage within the obstructed lumen. In one embodiment disclosed herein, the guidewire functionality is enhanced by an imaging system to guide, locate, and assess treatment.

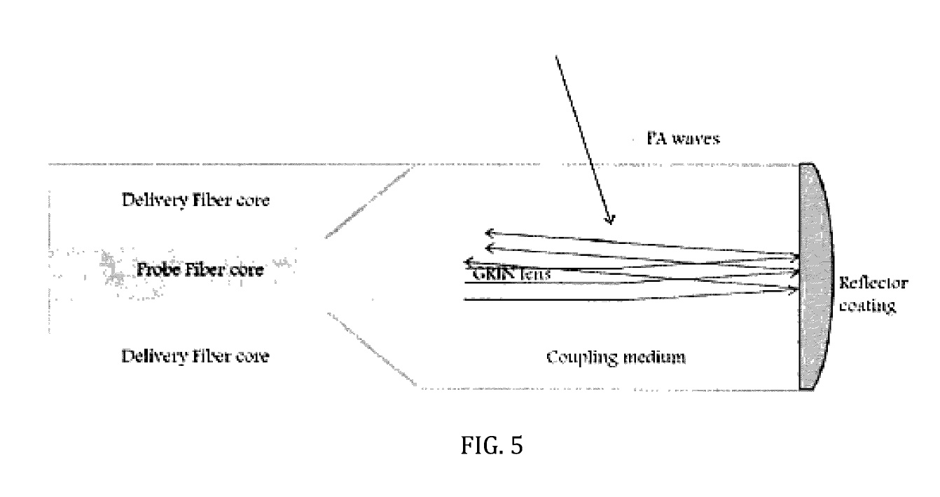

Certain embodiments integrate a photoacoustic probe into a catheter configuration by employing a fiber optic ultrasound sensor. A non-limiting example is illustrated in FIG. 5. In certain aspects, the probe can be coupled with the guidewire or guidewire like fiber. In some aspects, the device may be configured to deliver an excitation beam using a fiber optic bundle that is originally designed to deliver laser pulses for ablation. In certain embodiments, an optical fiber bundle is obtained by positioning a plurality of optical fibers in a columnar pattern around the internal circumference of the catheter body, and/or are positioned around the outer circumference of a core fiber. In some aspects, the core fiber is configured as a photoacoustic probe. In some aspects, the optical fibers can be configured in a circular or semicircular pattern.

In some aspects, the photoacoustic probe can comprise: the distal end of an optical fiber configured to transmit a probe beam; a lens which may be, but is not limited to a gradient index lens; a coupling medium, and/or a reflector. A non-limiting example of such a probe is shown in FIG. 5.

Gradient-index (GRIN) optics is a branch of optics that covers optical effects produced by a gradual variation of the refractive index of a material. Such variations may be used to produce lenses with flat surfaces, or lenses that do not have the aberrations typical of traditional spherical lenses. Gradient-index lenses may have a refraction gradient that is spherical, axial, or radial. The ability of GRIN lenses to have flat surfaces simplifies the mounting of the lens, which makes them useful where many very small lenses need to be mounted together, such as in photocopiers and scanners. The flat surface may also allow a GRIN lens to be easily fused to an optical fiber to, for example, produce collimated output.

In imaging applications, GRIN lenses are mainly used to reduce aberrations. The design of such lenses involves detailed calculations of aberrations as well as efficient manufacture of the lenses. A number of different materials have been used for GRIN lenses including optical glasses, plastics, germanium, zinc selenide, and sodium chloride. GRIN lenses can be made using various techniques that includes neutron irradiation—boron-rich glass is bombarded with neutrons to cause a change in the boron concentration, and thus the refractive index of the lens; chemical vapor deposition—involving the deposition of different glass with varying refractive indexes, onto a surface to produce a cumulative refractive change; partial polymerization—an organic monomer is partially polymerized using ultraviolet light at varying intensities to give a refractive gradient; ion exchange—glass is immersed into a liquid melt with lithium ions as a result of diffusion, sodium ions in the glass are partially exchanged with lithium ones, with a larger amount of exchange occurring at the edge, thus the sample obtains a gradient material structure and a corresponding gradient of the refractive index; ion Stuffing—phase separation of a specific glass causes pores to form, which can later be filled using a variety of salts or concentration of salts to give a varying gradient.

In certain aspects, the excitation light source is intended to irradiate the light of a specific wavelength to be absorbed by a specific component among the components of a target. In some embodiments, there is provided at least one pulsed light source that can generate pulsed light with a pulse width on the order of from several nanoseconds to several hundred nanoseconds. Non-limiting examples of light sources include a laser capable of obtaining a large output, light emitting diodes, and similar light sources. Various types of lasers can be used as a light source. Non-limiting examples include a solid-state laser, a gas laser, a dye laser, a semiconductor laser, and so on. In some aspects, the timing of irradiation, the waveform, the intensity, etc., of the laser are controlled by a signal processing device and/or a control unit. In certain aspects, the system can comprise at least one light or laser source that can be independently coupled to two or more optical fibers and provide at least two independently tunable beams. In certain aspects, two or more light sources can be employed. In some aspects, a light source provides at least two lasers, one laser may be pulsed and has a pulse width in a range of between about 1 nanosecond and about several hundred nanoseconds. In certain aspects, the wavelengths of the excitation beam is between about 10, 50, 100, 150, 200, 250, 300, 400, 500, 550, 600, 650 700 nm to about 600, 650, 700, 750, 800, 850, 900, 950, 1000, 1100, 1200, 1300, 1400, and 1500 nm, and all ranges therein. In certain aspects, the excitation laser is tuned to an absorption wave-length of a target to be imaged. Additional non-limiting examples of excitation light sources include Nd:YLF nanosecond lasers (e.g., Quantronix Falcon 527-30-M), Nd:YVO nanosecond lasers (e.g., Quantronix Lsprey-1064-20-L), or similar devices.

In some aspects, the device is capable of producing a probe beam. In some aspects, the probe beam can be supplied by a tunable diode laser. In some aspects, the probe beam is capable of deflection by acoustic waves. In some aspects, the wavelength is selective for the target. In some aspects, the probe beam has a wavelength range between 1200 nm and 1600 nm. A non-limiting example of a laser that may provide a probe beam is a new focus TLB-6600.

In some aspects, the device is capable of producing an ablation beam. In some aspects the ablation beam can be provided by a laser. In some aspects, the ablation beam produced is of a wavelength that is absorbed by the target to be ablated. In some aspects, the wavelength is selectively absorbed by the target to be ablated. Non-limiting examples of lasers that may provide an ablation beam includes: a XeCl laser at a wavelength of about 308 nanometers (nm) and an approximate pulse width of about 10 nanoseconds (nsec); and a high pulse energy ultraviolet excimer laser.

1. A device comprising an elongated catheter body having (i) a photoacoustic probe positioned at the distal end of the catheter body, the probe comprising an acoustic chamber comprising an acoustic coupling medium, (ii) a reflector capping the distal end of the acoustic chamber, (iii) a first optical fiber positioned along or parallel to the central axis of the catheter body and terminating at the proximal end of the acoustic chamber and configured to direct a probe beam across the acoustic chamber to the reflector which is configured to return the probe beam to the first optical fiber.

2. The device of claim 1, wherein the photoacoustic probe is a photoacoustic ultrasound probe.

3. The device of claim 1, wherein the probe is configured to be an all-optical probe.

4. The device of claim 1, wherein the first optical fiber is a single mode or multi-mode optical fiber.

5-6. (canceled)

7. The device of claim 1, wherein the probe further comprises a lens.

8. The device of claim 7, wherein the lens is a gradient-index lens.

9-10. (canceled)

11. The device of claim 1, wherein a wavelength of the probe beam is between 1200 nm to 1600 nm.

12. The device of claim 1, wherein a detector is coupled to a proximal end of the first optical fiber.

13. The device of claim 12, wherein the detector is an interferometer.

14. The device of claim 1, further comprising at least a second optical fiber parallel to the first optical fiber, which is configured to provide a second beam.

15. The device of claim 14, wherein the second beam is an excitation beam.

16-17. (canceled)

18. The device of claim 15, wherein the excitation beam is capable of being pulsed with a pulse width of between 1 nanosecond to 1 microsecond.

19. (canceled)

20. The device of claim 14, wherein the second beam is an ablation beam.

21. (canceled)

22. The device of claim 20, wherein the ablation beam is a laser beam.

23-24. (canceled)

25. The device of claim 14, wherein the second fiber is configured to provide an excitation beam and an ablation beam.

26. The device of claim 14, further comprising at least one additional optical fiber circumferential to the first optical fiber, wherein at least one of the at least one additional optical fiber is configured to provide an additional beam.

27. The device of claim 26, wherein at least one of the at least one additional optical fiber is configured to provide an excitation beam.

28. The device of claim 26, wherein at least one of the at least one additional optical fiber is configured to provide an ablation beam.

29. (canceled)

30. The device of claim 1, wherein the device is configured to image 360° with no need for physical rotation of the catheter.

31-33. (canceled)

34. A catheter system comprising:
at least one excitation and/or ablation beam source;
a probe beam source;
an optical/fiber optics interface;
an interferometer/optical coherence detector;
a control system configured to image and provide treatment to a tissue target; and
a vascular intervention device comprising:
(i) an elongated catheter body;
(ii) a photoacoustic probe positioned at the distal end of the catheter body, the probe comprising an acoustic chamber comprising an acoustic coupling medium,
(iii) a reflector capping the distal end of the acoustic chamber,
(iv) a first optical fiber positioned along the central axis of the catheter body and terminating at the proximal end of the acoustic chamber and configured to direct a probe beam across the acoustic chamber to the reflector which is configured to return the probe beam to the first optical fiber, and
(v) at least a second optical fiber parallel to the first optical fiber, wherein the second optical fiber is configured to provide a second beam;
wherein the at least one excitation and/or ablation beam source and the probe beam source are coupled to the vascular intervention device via the optical/fiber optics interface, and
wherein the at least one excitation and/or ablation beam source, probe beam source, and interferometer/optical coherence detector are coupled to the control system configured to image and provide treatment to a tissue target.

35-69. (canceled)
